(19) United States
(12) Patent Application Publication
(10) Pub. No.: US 2025/0082526 A1
(43) Pub. Date: Mar. 13, 2025

(54) PRESSURE MEASURING DEVICE, CONTROL DEVICE OF IRRADIATION DEVICE AND CONTROL SYSTEM OF PRESSURE MEASUREMENT BASED IRRADIATION DEVICE

(71) Applicant: JEISYS MEDICAL INC., Seoul (KR)

(72) Inventors: Dong Hwan Kang, Seoul (KR); Sun Kim, Seoul (KR); Hyun Sook Lee, Seoul (KR); Jung Su Jeon, Seoul (KR); Dae Keon Woo, Seoul (KR)

(73) Assignee: JEISYS MEDICAL INC., Seoul (KR)

(21) Appl. No.: 18/882,983

(22) Filed: Sep. 12, 2024

(30) Foreign Application Priority Data
Sep. 12, 2023 (KR) 10-2023-0120897

(51) Int. Cl. A61G 7/05

(52) U.S. Cl. CPC A61G 7/0524

(57) ABSTRACT

A system includes: a pressure measuring device configured to measure an amount of pressure on a bed; an irradiation device configured to press on a body of a user lying on the bed and to irradiate energy towards the user; and a control device configured to control the irradiation device, wherein the control device is configured to: obtain a pressure measurement from the pressure measuring device; and control the irradiation device to stop an operation of the irradiation device based on the pressure measurement exceeding a preset level.

### CROSS-REFERENCE TO RELATED APPLICATIONS

This application claims benefit to Korean Patent Application No. KR 10-2023-0120897, filed on Sep. 12, 2023, which is hereby incorporated by reference herein.

### FIELD

The present disclosure relates to a pressure measuring device, a control device for an irradiation device, and a control system for an irradiation device based on pressure measurement.

### BACKGROUND

Conventionally, a device that irradiates energy and presses on a human body includes a device that burns and removes specific subcutaneous tissues such as tumors in the skin by using the heat generated by high-intensity focused ultrasound (HIFU), or that generates heat by high-frequency output to skin tissues to cause denaturation and contraction of collagen fibers.

However, when irradiating energy while pressing on the human body, the conventional irradiation device has limitations in improving the accuracy of the irradiation device, which limits the prevention of safety accidents and the prevention of damage to the irradiation device.

### SUMMARY

In an exemplary embodiment, the present disclosure provides a system. The system includes: a pressure measuring device configured to measure an amount of pressure on a bed; an irradiation device configured to press on a body of a user lying on the bed and to irradiate energy towards the user; and a control device configured to control the irradiation device, wherein the control device is configured to: obtain a pressure measurement from the pressure measuring device; and control the irradiation device to stop an operation of the irradiation device based on the pressure measurement exceeding a preset level.

In another exemplary embodiment, the present disclosure provides a control device for an irradiation device. The control device includes: a receiver configured to receive a communication from a pressure measuring device; and a processor electrically connected to the receiver, wherein the processor is configured to: obtain a pressure measurement from the pressure measuring device via the receiver, determine whether the pressure measurement exceeds a preset value, and control the irradiation device to stop an operation of the irradiation device based on the pressure of the bed exceeding the preset level, wherein the operation of the irradiation device includes applying pressure to a body of a user lying on the bed and irradiating the body of the user with energy.

In yet another exemplary embodiment, the present disclosure provides a method for controlling an irradiation device. The method includes: obtaining, by a control device of the irradiation device, a pressure measurement corresponding to an amount of pressure on a bed from a pressure measuring device; determining, by the control device, whether the pressure measurement exceeds a preset level; and controlling, by the processor, the irradiation device to stop an operation of the irradiation device based on the pressure of the bed exceeding the preset level, wherein the operation of the irradiation device includes applying pressure to a body of a user lying on the bed and irradiating the body of the user with energy.

### BRIEF DESCRIPTION OF THE FIGURES

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a diagram illustrating a control system of an irradiation device based on pressure measurement according to an exemplary embodiment of the present disclosure.

FIG. 2 is a diagram illustrating a configuration of the pressure measuring device shown in FIG. 1.

FIG. 3 is a diagram illustrating a configuration of the control device shown in FIG. 1.

FIG. 4 is a diagram illustrating a configuration of the irradiation device shown in FIG. 1.

FIGS. 5 to 17 are diagrams illustrating a method for controlling an irradiation device based on pressure measurement according to exemplary embodiments of the present disclosure.

### DETAILED DESCRIPTION

Exemplary embodiments of the present disclosure improve the accuracy of an irradiation device that applies pressure to a human body and irradiates energy, thereby preventing safety accidents and preventing damage to the irradiation device.

It will be appreciated that technical problems addressed by exemplary embodiments of the present disclosure are not limited to the technical problems mentioned above, and other technical problems not mentioned may also be addressed.

In an aspect of the present disclosure, a pressure measuring device may include a measuring unit (e.g., a measuring device such as a sensor) disposed on a side of a bed and configured to measure a pressure on the bed (e.g., in terms of a weight or force pressing down on the bed); and a processor configured to transmit the pressure measurement to a control device (e.g., a controller) of an irradiation device through a communication unit (e.g., a transmitter).

Furthermore, the measuring unit may be disposed on a moving frame of the bed.

Furthermore, the measuring unit may be disposed on a support frame of the bed.

Furthermore, the measuring unit may be further disposed on a side of the irradiation device that presses on a body of a user lying on the bed and irradiates energy.

Furthermore, the measuring unit may include a load cell.

Furthermore, in another aspect of the present disclosure, a control device of an irradiation device may include a communication unit (e.g., a receiver) configured to perform communication with a pressure measuring device; and a processor electrically connected to the communication unit, wherein the processor is configured to: receive a pressure measurement corresponding to an amount of pressure on a bed measured from the pressure measuring device through the communication unit, and control the irradiation device to stop an operation of the irradiation device that presses on a body of a user lying on the bed and irradiates energy, based on the pressure measurement exceeding a preset level.

Furthermore, when determining whether the pressure measurement exceeds the preset level, the processor may be configured to determine whether a weight change on the bed due to the pressure of the body on the bed is greater than a preset reference value.

Furthermore, when determining whether the pressure of the bed exceeds the preset level, the processor may be configured to further determine whether a weight change corresponding to the irradiation device due to the pressure on the body is greater than a preset reference value.

Furthermore, in still another aspect of the present disclosure, a method for controlling an irradiation device, performed by a control device may include receiving a pressure measurement corresponding to an amount of pressure on a bed measured from the pressure measuring device through a communication unit of the control device; determining, by a processor of the control device, whether the pressure measurement exceeds a preset level; and controlling, by the processor, the irradiation device to stop an operation of the irradiation device that presses on a body of a user lying on the bed and irradiates energy, based on the pressure measurement exceeding a preset level.

Furthermore, in still another aspect of the present disclosure, a system for controlling an irradiation device based on pressure measurement may include a pressure measuring device configured to measure an amount of pressure on a bed; an irradiation device configured to press on a body of a user lying on the bed and irradiate energy; and a control device configured to control the irradiation device, wherein the control device is configured to: receive a pressure measurement corresponding to an amount of pressure on a bed measured from the pressure measuring device, and control the irradiation device to stop an operation of the irradiation device, based on the pressure measurement exceeding a preset level.

In addition, a computer program stored in a non-transitory computer-readable recording medium may be further provided to perform a method of controlling an irradiation device by being combined with a computer as hardware.

In addition, a non-transitory computer-readable recording medium recording a computer program for executing a method for implementing the present disclosure may be further provided.

In the drawings, the same reference numeral refers to the same element. This disclosure may not describe all elements of embodiments, and general knowledge known to a person of ordinary skill in the art or repeated contents of the embodiments may be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network. Furthermore, when a certain part “includes” a certain element, other elements are not excluded unless explicitly described otherwise, and other elements may in fact be included.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

Hereinafter, operation principles and embodiments of the present disclosure will be described with reference to the accompanying drawings.

A control device according to the present disclosure may include various devices that can perform computational processing and provide results to a user. For example, a device according to the present disclosure may include a computer, a server device, and/or a portable terminal, or may be in the form of one of them.

Here, the computer may include, for example, a notebook, a desktop, a laptop, a tablet PC, a slate PC, or the like having a web browser installed.

The server device may be a server that communicates with an external device to process information, and may include an application server, a computing server, a database server, a file server, a mail server, a proxy server, or a web server.

The portable terminal may be a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminals, smart phones, and the like, and wearable devices such as watches, rings, bracelets, anklets, necklaces, glasses, contact lenses, or head-mounted devices (HMDs).

A control system of an irradiation device based on pressure measurement according to the present disclosure may include a pressure measuring device configured to measure an amount of pressure on a bed; an irradiation device configured to press on a body of a user lying on the bed and irradiate energy; and a control device configured to control the irradiation device, and the control device may receive a pressure measurement corresponding to an amount of pressure on a bed measured from the pressure measuring device, and control the irradiation device to stop an operation of the irradiation device, based on the pressure measurement exceeding a preset level.

The control system of the irradiation device based on pressure measurement

according to the present disclosure can improve the accuracy of the irradiation device that irradiates energy and applies pressure to a human body, thereby preventing safety accidents and preventing damage to the irradiation device.

Hereinafter, the control system of the irradiation device based on pressure measurement according to the present disclosure will be examined in detail.

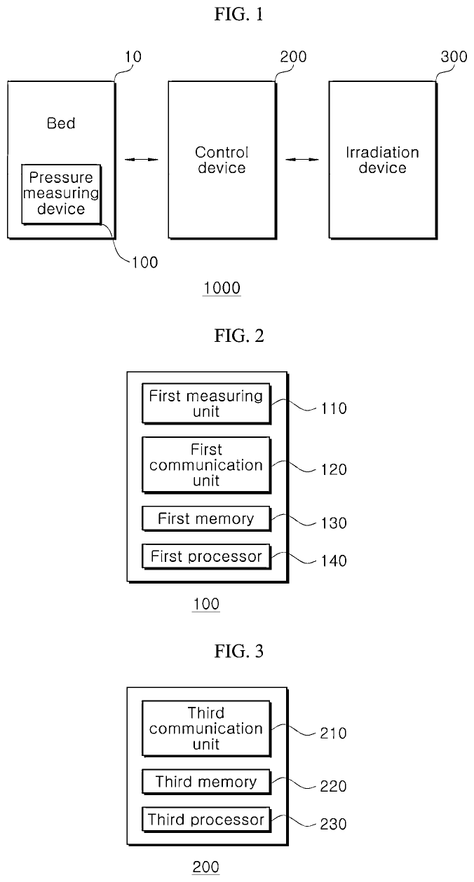

FIG. 1 is a diagram illustrating a control system of an irradiation device based on pressure measurement according to the present disclosure. FIG. 2 is a diagram illustrating a configuration of the pressure measuring device shown in FIG. 1.

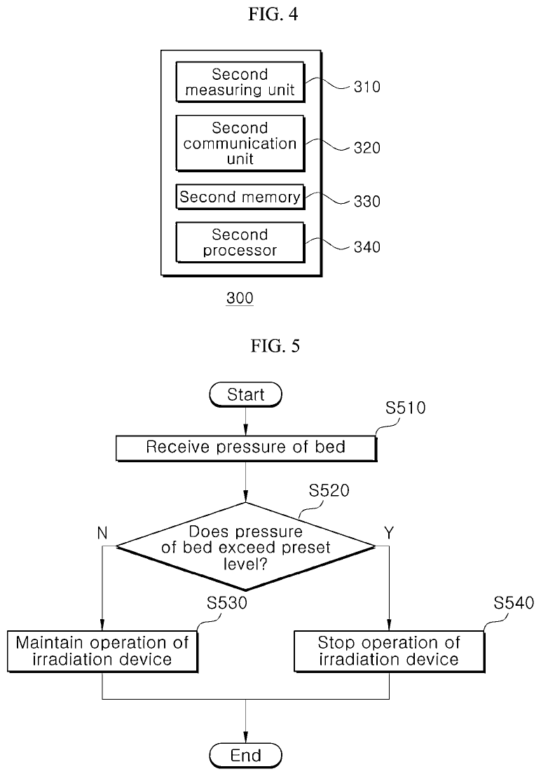

FIG. 3 is a diagram illustrating a configuration of the control device shown in FIG. 1, and FIG. 4 is a diagram illustrating a configuration of the irradiation device shown in FIG. 1.

Referring to FIG. 1 to FIG. 4, a control system 1000 of an irradiation device based on pressure measurement may include a pressure measuring device 100, a control device 200, and an irradiation device 300.

The pressure measuring device 100 may measure an amount of pressure on a bed 10. In this case, the pressure measuring device 100 may include a first measuring unit 110, a first communication unit 120, a first memory 130, and a first processor 140.

The first measuring unit 110 is disposed on a side of the bed 10 and may measure an amount of pressure on the bed 10.

For example, the first measuring unit 110 may be disposed on a moving frame of the bed 10. For another example, the first measuring unit 110 may be disposed on a support frame of the bed 10. In this case, the first measuring unit 110 may be a load cell.

The first communication unit 120 may perform communication with the first measuring unit 110. The first communication unit 120 may transmit the pressure measurement measured through the first measuring unit 110 to the first memory 130. Here, the first memory 130 may store data related to the amount of pressure on the bed 10.

The first processor 140 may control an operation based on an amount of pressure on the bed 10. The first processor 140 may transmit the pressure measurement stored in the first memory 130 to the control device 200 of the irradiation device 300 through the first communication unit 120.

The irradiation device 300 may measure an amount of pressure on the bed 10. The irradiation device 300 may include a second measuring unit 310, a second communication unit 320, a second memory 330, and a second processor 340.

The second measuring unit 310 is provided on one side of the irradiation device 300 and may measure an amount of pressure on the bed 10. The second measuring unit 310 may be a load cell.

The second communication unit 320 may communicate with the second measurement unit 310. The second communication unit 320 may transmit the pressure measurement measured through the second measurement unit 310 to the second memory 330. Here, the second memory 330 may store data related to the pressure measurement.

The second processor 340 may control an operation based on the pressure measurement. The second processor 340 may transmit the pressure measurement stored in the second memory 330 to the control device 200 of the irradiation device 300 through the second communication unit 320.

The control device 200 may include a third communication unit 210, a third memory 220, and a third processor 230.

The third communication unit 210 may perform communication with the first communication unit 120 of the pressure measuring device 100. The third communication unit 210 may receive the pressure measurement from the first measurement unit 110 through the first communication unit 120 and transmit the received pressure measurement to the third memory 220. Here, the third memory 220 may store data related to the pressure measurement.

The third processor 230 may control the operation of the irradiation device 300 that presses on the body of the user lying on the bed 10 and irradiates energy. The third processor 230 may control the irradiation device 300 to stop the operation of the irradiation device 300 when the pressure measurement exceeds a preset level.

Here, when determining whether the pressure measurement exceeds a preset level, the third processor 230 may determine whether a weight change on the bed 10 due to pressure of the human body on the bed is greater than or equal to a preset reference value.

As an example, the third processor 230 may control the irradiation device 300 to stop the operation of the irradiation device 300 in the case that the weight change on the bed 10 due to the pressure of the body on the bed is greater than or equal to a preset reference value.

As another example, the third processor 230 may control the irradiation device 300 to maintain the operation of the irradiation device 300 in the case that the weight change on the bed 10 due to the pressure of the body on the bed is less than a preset reference value. The third communication unit 210 may communicate with the second

communication unit 320 of the irradiation device 300. The third communication unit 210 may receive the pressure measurement measured from the second measurement unit 310 through the second communication unit 320 and transmit the received pressure measurement to the third memory 220. Here, the third memory 220 may store data related to the pressure measurement.

The third processor 230 may control the operation of the irradiation device 300 that presses on the body of the user lying on the bed 10 and irradiates energy. The third processor 230 may control the irradiation device 300 to stop the operation of the irradiation device 300 when the pressure measurement exceeds a preset level.

Here, when the third processor 230 determines whether the pressure measurement exceeds a preset level, the third processor 230 may determine whether a weight change corresponding to the irradiation device 300 pressing on the body is greater than or equal to a preset reference value.

As an example, the third processor 230 may control the irradiation device 300 to stop the operation of the irradiation device 300 in the case that the weight change corresponding to the irradiation device 300 is greater than or equal to a preset reference value.

As another example, the third processor 230 may control the irradiation device 300 to maintain the operation of the irradiation device 300 in the case that a weight change corresponding to the irradiation device 300 applying pressure to the human body is less than a preset reference value.

FIGS. 5 to 17 are diagrams illustrating a method for controlling an irradiation device based on pressure measurement according to the present disclosure.

Referring to FIGS. 5 to 17, the method for controlling an irradiation device based on pressure measurement may include a receiving step S510, a determining step S520, a maintaining step S530, and a stopping step S540.

A communication unit may receive a pressure measurement corresponding to an amount of pressure on the bed 10 measured from the pressure measuring device 100 (step S510).

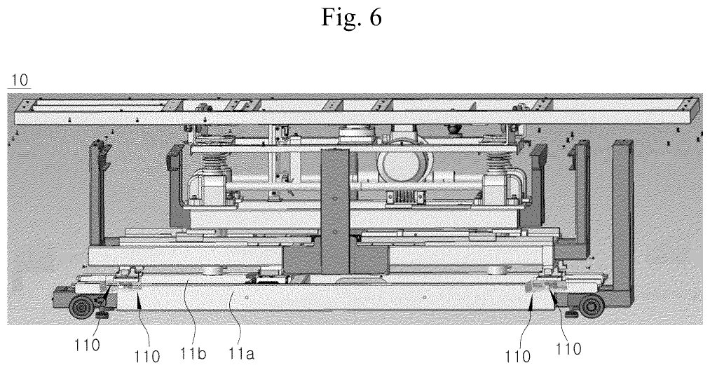

For example, as illustrated in FIG. 6, the first measuring unit 110 may be disposed on moving frames 11a and 11b of the bed 10 and may measure an amount of the pressure on the bed 10. The first measuring unit 110 may be a load cell. For example, the number of first measuring units 110 may be four, and the four first measuring units 110 may be provided at opposite corners of the moving frames 11a and 11b for accurate pressure measurement. In this case, the four first measuring units 110 may be provided on any one of upper, lower, left, and right sides of the moving frames 11a and 11b for accurate pressure measurement while considering the installation space with respect to external conditions. These four first measuring units 110 may each measure an amount of pressure on the bed 10. That is, in the areas formed by connecting the positions of the four first measuring units 110, the four first measuring units 110 may respectively accurately measure the amount of pressure on the bed 10.

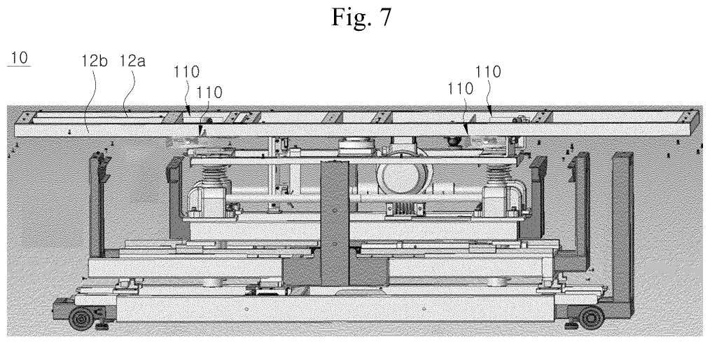

As another example, as shown in FIG. 7, the first measuring unit 110 may be disposed on the support frames 12a and 12b of the bed 10 and may measure an amount of pressure on the bed 10. The first measuring unit 110 may be a load cell. For example, the number of first measuring units 110 may be four, and the four first measuring units 110 may be provided on opposite corners of the support frames 12a and 12b for accurate pressure measurement. The four first measuring units 110 may be provided on any one of upper, lower, left, and right sides of the support frames 12a and 12b for accurate pressure measurement while considering the installation space with respect to external conditions. These four first measuring units 110 may each measure an amount of pressure on the bed 10. That is, in the areas formed by connecting the positions of the four first measuring units 110, the four first measuring units 110 may respectively accurately measure an amount of pressure on the bed 10.

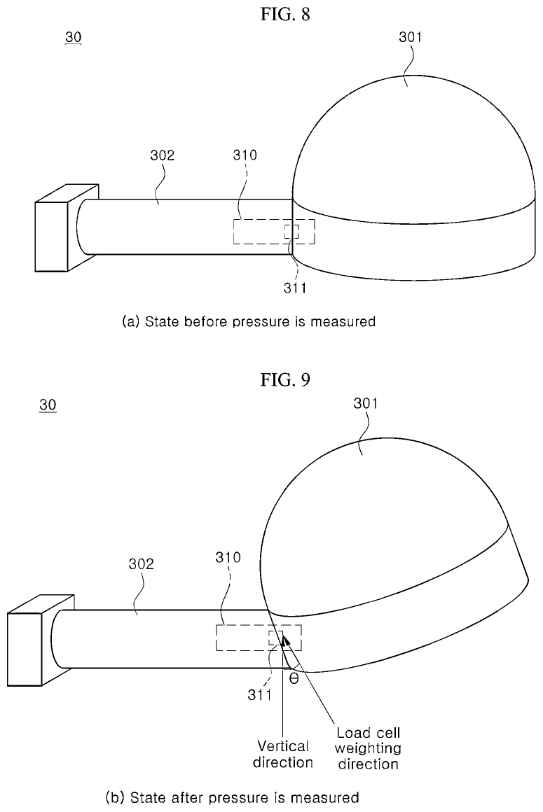

As another example 30, as shown in FIGS. 8 and 9, the second measuring unit 310 may be provided on a side of the irradiation device 300 and may measure an amount of pressure on the bed 10. The second measuring unit 310 may be provided between a support 302 connected to an irradiation head 301. Here, the second measuring unit 310 may be provided between an inside of the support 302 connected to the irradiation head 301 or on the outside of the support 302 connected to the irradiation head 301 for accurate pressure measurement while considering the installation space with respect to external conditions. The second measuring unit 310 may be a load cell. When a sensing module 311 is turned on, the second measuring unit 310 may be electrically connected to the irradiation head 301, and when the irradiation head 301 presses on the user lying on the bed 10, the degree of distortion of the load cell between the descending support 302 and the bent irradiation head 301 may be measured. In other words, when a pressure is applied to the sensing module 311 in a vertical direction, the irradiation head 301 performs a tilt function and a rotation function, so the pressure generated on the tilted irradiation head 301 may be attenuated by the tilted angle θ. That is, the second measuring unit 310 may calculate the value for the vertical direction by multiplying a cosine value by the value for the load cell weighting direction based on the trigonometric function. The third processor 230 may determine whether an amount of pressure on the bed 10 exceeds a preset level (step S520).

In the case that the amount of pressure on the bed 10 does not exceed the preset level, the third processor 230 may control the irradiation device 300 to maintain the operation of the irradiation device 300 that presses on the body of the user lying on the bed 10 and irradiates energy (step S530). The irradiation device 300 may be a device that presses on the body of the user lying on the bed 10 and irradiates at least one of high-frequency energy or ultrasonic energy. The irradiation device 300 may also be a device that irradiates energy of a different level.

The third processor 230 may control the irradiation device 300 to stop the operation of the irradiation device 300 when the amount of pressure on the bed 10 exceeds a preset level (step S540).

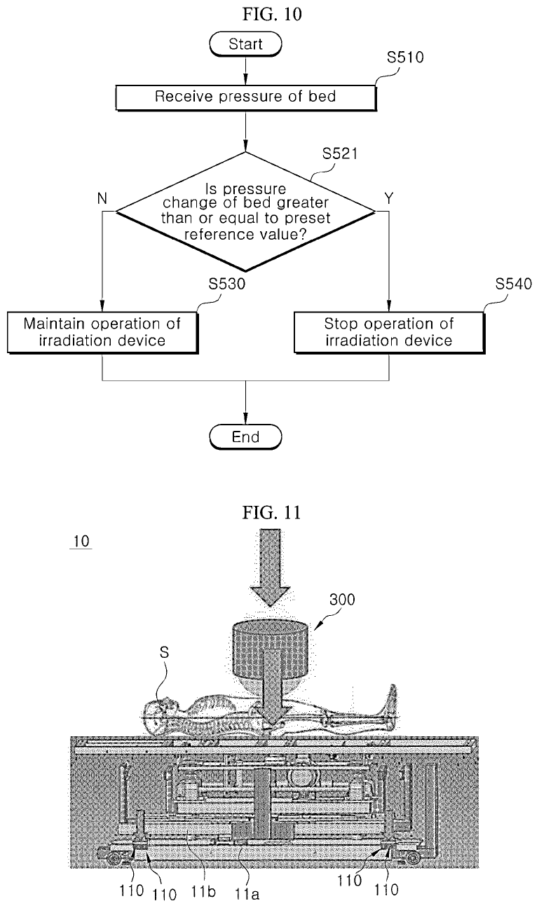

For example, as shown in FIG. 10, in the case of determining whether the amount of pressure on the bed 10 exceeds a preset level, the third processor 230 may determine whether a weight change on the bed 10 due to a human body pressing on the bed is greater than or equal to a preset reference value (step S521).

For example, as shown in FIG. 11, when the body of the user S lying on the bed 10 is pressed on by the irradiation device 300, the third processor 230 may determine whether the weight change on the bed 10 measured through the first measuring unit 110 provided on the moving frames 11a and 11b of the bed 10 is greater than or equal to a preset reference value. In this case, the third processor 230 may control the irradiation device 300 to maintain the operation of the irradiation device 300 that presses on the body of the user lying on the bed 10 and irradiates energy in the case that the weight change amount on the bed 10 is less than the preset reference value.

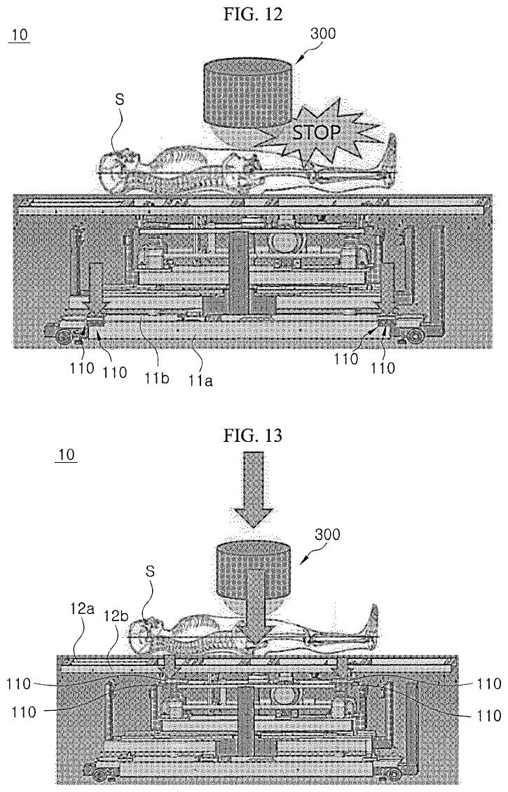

In addition, as illustrated in FIG. 12, the third processor 230 may control the irradiation device 300 to stop the operation of the irradiation device 300 in the case that the weight change amount on the bed 10 is greater than the preset reference value.

The third processor 230 may receive the respective weight change amounts measured through the four first measuring units 110 provided in the moving frames 11a and 11b, calculate an average value for the received weight change amounts, and may further determine whether the calculated average value is greater than or equal to a preset reference average value. In the case that the calculated average value is less than or equal to the preset reference average value, the third processor 230 may further control the irradiation device 300 to maintain the operation of the irradiation device 300. In addition, in the case that the calculated average value is greater than or equal to the preset reference average value, the third processor 230 may further control the irradiation device 300 to stop the operation of the irradiation device 300.

For another example, as illustrated in FIG. 13, when the irradiation device 300 presses on the body of the user S lying on the bed 10, the third processor 230 may determine whether a weight change on the bed 10 measured through the first measuring unit 110 provided on the support frames 12a and 12b of the bed 10 is greater than or equal to a preset reference value. In the case that the weight change of the bed 10 is less than the preset reference value, the third processor 230 may control the irradiation device 300 to maintain the operation of the irradiation device 300.

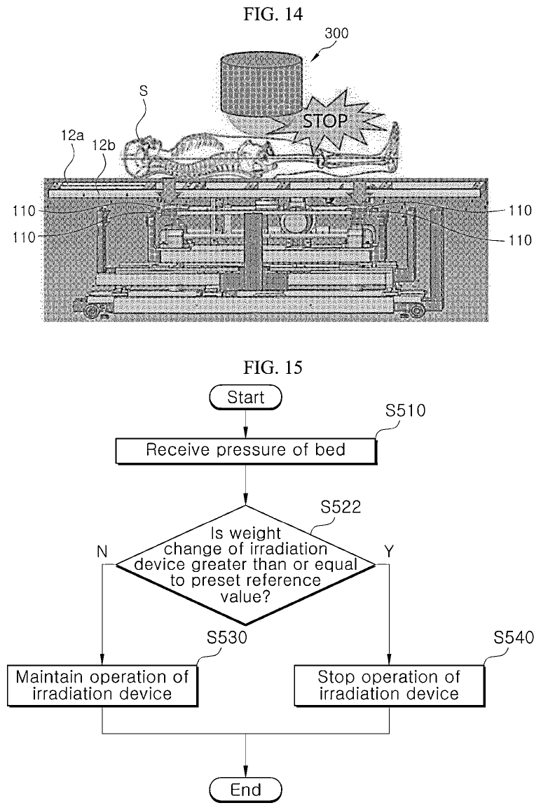

In addition, as illustrated in FIG. 14, the third processor 230 may control the irradiation device 300 to stop the operation of the irradiation device 300 in the case that the weight change amount on the bed 10 is greater than or equal to a preset reference value.

The third processor 230 may receive the respective weight change amount on the bed 10 measured through four first measuring units 110 provided on the support frames 12a and 12b, calculate an average value for the received weight change amounts, and may further determine whether the calculated average value is greater than or equal to a preset reference average value. In the case that the calculated average value is less than the preset reference average value, the third processor 230 may further control the irradiation device 300 to maintain the operation of the irradiation device 300. In addition, in the case that the calculated average value is greater than the preset reference average value, the third processor 230 may further control the irradiation device 300 to stop the operation of the irradiation device 300.

As another example, as illustrated in FIG. 15, when determining whether the pressure on the bed 10 exceeds the preset level, the third processor 230 may determine whether a weight change corresponding to the irradiation device 300 applying pressure on the body is greater than or equal to the preset reference value (step S522).

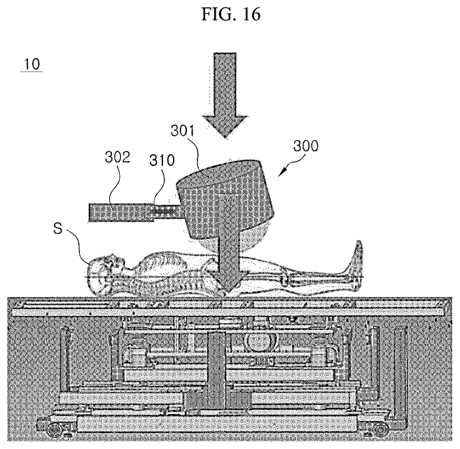

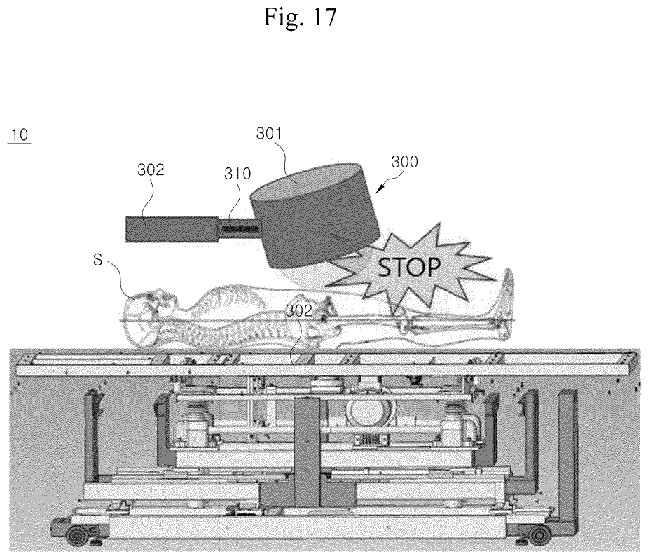

For example, as illustrated in FIG. 16, when the body of the user S lying on the bed 10 is pressed on by the irradiation device 300, the third processor 230 may determine whether a weight change corresponding to the irradiation device 300 measured through the second measuring unit 310 provided on one side of the irradiation device 300 is greater than or equal to a preset reference value. In the case that the weight change of the irradiation device 300 is less than the preset reference value, the third processor 230 may control the irradiation device 300 to maintain the operation of the irradiation device 300.

In addition, as illustrated in FIG. 17, the third processor 230 may control the irradiation device 300 to stop the operation of the irradiation device 300 in the case that the weight change of the irradiation device 300 is greater than or equal to the preset reference value.

The first to third memories 130, 220, and 330 may store data for an algorithm for controlling the operation of components in the device or a program that reproduces the algorithm, respectively. The first to third processors 140, 230, and 340 may perform the above-described operation using the data stored in the first to third memories 130, 220, and 330, respectively. Here, the first to third memories 130, 220, and 330 and the first to third processors 140, 230, and 340 may be implemented as separate chips, respectively. In addition, the first to third memories 130, 220, and 330 and the first to third processors 140, 230, and 340 may be implemented as a single chip.

The first to third memories 130, 220, and 330 may store data supporting various functions of the device, programs for the operation of components within the device, may store input/output data, and may store a plurality of application programs or applications executed on the device, data for the operation of the device, and commands. At least some of these application programs may be downloaded from an external server via wireless communication.

The first to third memories 130, 220, and 330 may include at least one type of storage medium among a flash memory type, a hard disk type, an SSD type, an SDD type, a multimedia card micro type, a card type memory (e.g., an SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk.

The first to third communication units 120, 210, and 320 may include at least one of a wired communication module or a wireless communication module.

The wired communication module may include various wired communication modules such as a Local Area Network (LAN) module, a Wide Area Network (WAN) module, or a Value Added Network (VAN) module, as well as various cable communication modules such as a Universal Serial Bus (USB), a High Definition Multimedia Interface (HDMI), a Digital Visual Interface (DVI), RS-232 (recommended standard232), power line communication, or a plain old telephone service (POTS).

The wireless communication module may include a wireless communication module that supports various wireless communication schemes such as a WiFi module, a Wireless broadband module, and a GSM (global System for Mobile Communication), CDMA (Code Division Multiple Access), WCDMA (Wideband Code Division Multiple Access), UMTS (universal mobile telecommunications system), TDMA (Time Division Multiple Access), LTE (Long Term Evolution), 4G, 5G, and 6G.

It will be appreciated that exemplary embodiments of the present disclosure provide for preventing safety accidents and prevent damage to the irradiation device by improving the accuracy of the irradiation device.

At least one component may be added or deleted in accordance with the performance of the components illustrated in FIGS. 1 to 4, FIGS. 6 to 9, FIGS. 11 to 14, FIGS. 16, and FIGS. 17. In addition, it will be readily understood by a person of ordinary skill in the art that the reciprocal positions of the components may be changed in accordance with the performance or structure of the system.

Although FIG. 5, FIG. 10, and FIG. 15 describe sequential execution of the multiple steps, this is merely an exemplary implementation, and a person of ordinary skill in the art may modify and change the order described in FIG. 5, FIG. 10, and FIG. 15 without departing from the principles of the present disclosure, or may modify and change the order described in FIG. 5, FIG. 10, and FIG. 15 in various ways by executing one or more of the multiple steps in parallel, and thus FIG. 5, FIG. 10, and FIG. 15 are not limited to a specific sequential order.

It will be appreciated that, according to exemplary embodiments of the present disclosure, the accuracy of an irradiation device is improved, thereby preventing safety accidents and preventing damage to the irradiation device. It will be appreciated that effects and advantages of the present disclosure are not limited to the effects and advantages mentioned above.

It will be appreciated that exemplary embodiments of the present disclosure may be implemented in the form of a recording medium that stores instructions executable by a computer. The instructions may be stored in the form of program codes, and when executed by a processor, may generate a program module to perform the operations of the disclosed embodiments. The recording medium may be implemented as a non-transitory computer-readable recording medium.

The computer-readable recording medium includes all types of recording media that store instructions that can be decoded by a computer. For example, there may be a ROM (Read Only Memory), a RAM (Random Access Memory), a magnetic tape, a magnetic disk, a flash memory, an optical data storage device, and the like.

The disclosed embodiments have been described with reference to the attached drawings as described above. It will be appreciated that the present disclosure can be implemented in a form other than the disclosed embodiments without departing from the principles of the present disclosure. The disclosed embodiments are exemplary and should not be construed as limiting.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A system, comprising:
a pressure measuring device configured to measure an amount of pressure on a bed;
an irradiation device configured to press on a body of a user lying on the bed and to irradiate energy towards the user; and
a control device configured to control the irradiation device, wherein the control device is configured to:
obtain a pressure measurement from the pressure measuring device; and
control the irradiation device to stop an operation of the irradiation device based on the pressure measurement exceeding a preset level.

2. The system according to claim 1, wherein the pressure measuring device comprises:
a sensor disposed on a side of a bed and configured to generate the pressure measurement; and
a transmitter configured to transmit the pressure measurement to the control device.

3. The system of claim 1, wherein the measuring device is disposed on a moving frame of the bed.

4. The system of claim 1, wherein the measuring device is disposed on a support frame of the bed.

5. The system of claim 1, wherein the measuring device is disposed on a side of the irradiation device.

6. The system of claim 1, wherein the measuring device includes a load cell.

7. The system of claim 1, wherein the control device is further configured to determine whether the pressure measurement exceeds the preset level.

8. The system of claim 7, wherein determining whether the pressure measurement exceeds the preset level comprises determining whether a weight change of the bed due to pressure of the body on the bed is greater than a preset reference value.

9. The control device of claim 7, wherein determining whether the pressure measurement exceeds the preset level comprises determining whether a weight change corresponding to the irradiation device applying pressure on the body is greater than a preset reference value.

10. A control device for an irradiation device, comprising:
a receiver configured to receive a communication from a pressure measuring device; and
a processor electrically connected to the receiver, wherein the processor is configured to:
obtain a pressure measurement from the pressure measuring device via the receiver,
determine whether the pressure measurement exceeds a preset value, and
control the irradiation device to stop an operation of the irradiation device based on the pressure of the bed exceeding the preset level, wherein the operation of the irradiation device includes applying pressure to a body of a user lying on the bed and irradiating the body of the user with energy.

11. The control device of claim 10, wherein determining whether the pressure measurement exceeds the preset level comprises determining whether a weight change of the bed due to pressure of the body on the bed is greater than a preset reference value.

12. The control device of claim 10, wherein determining whether the pressure measurement exceeds the preset level comprises determining whether a weight change corresponding to the irradiation device applying pressure on the body is greater than a preset reference value.

13. A method for controlling an irradiation device, comprising:
obtaining, by a control device of the irradiation device, a pressure measurement corresponding to an amount of pressure on a bed from a pressure measuring device;
determining, by the control device, whether the pressure measurement exceeds a preset level; and
controlling, by the processor, the irradiation device to stop an operation of the irradiation device based on the pressure of the bed exceeding the preset level, wherein the operation of the irradiation device includes applying pressure to a body of a user lying on the bed and irradiating the body of the user with energy.

14. The method of claim 13, wherein determining whether the pressure measurement exceeds the preset level comprises determining whether a weight change of the bed due to pressure of the body on the bed is greater than a preset reference value.

15. The method of claim 13, wherein determining whether the pressure measurement exceeds the preset level comprises determining whether a weight change corresponding to the irradiation device applying pressure on the body is greater than a preset reference value.

16. The method of claim 13, wherein the pressure measuring device comprises:
a sensor disposed on a side of a bed and configured to generate the pressure measurement; and
a transmitter configured to transmit the pressure measurement to the control device.

17. The method of claim 13, wherein the measuring device is disposed on a moving frame of the bed.

18. The method of claim 13, wherein the measuring device is disposed on a support frame of the bed.

19. The method of claim 13, wherein the measuring device is disposed on a side of the irradiation device.

20. The method of claim 13, wherein the measuring device includes a load cell.
